(19) United States
(12) Patent Application Publication
(10) Pub. No.: US 2021/0287801 A1
(43) Pub. Date: Sep. 16, 2021

(54) METHOD FOR PREDICTING DISEASE STATE, THERAPEUTIC RESPONSE, AND OUTCOMES BY SPATIAL BIOMARKERS

(71) Applicant: BIOSYNTAGMA, INC., Phoenix, AZ (US)

(72) Inventors: David Wayne Richardson, Chandler, AZ (US); Dmitry Derkach, Gilbert, AZ (US); Colleen Ziegler, Tempe, AZ (US); Chris DeSilva, Tempe, AZ (US); Isaiah Slemons, Phoenix, AZ (US)

(73) Assignee: BIOSYNTAGMA, INC., Phoenix, AZ (US)

(21) Appl. No.: 17/198,822

(22) Filed: Mar. 11, 2021

Related U.S. Application Data
(60) Provisional application No. 62/988,341, filed on Mar. 11, 2020.

(51) Int. Cl. G16H 50/20; G16H 50/30; G16H 50/70; G16B 20/40; G16H 30/40

(52) U.S. Cl. CPC G16H 50/20; G16H 50/30; G16H 30/40; G16B 20/40; G16H 50/70

(57) ABSTRACT

Provided herein are methods of processing in-situ (spatial) molecular data from comics measurements of solid tissues to identify complex, network-level biomarkers using deep learning based on location, molecular analyte, biological interactions, and patient metadata to classify disease states, identify drug targets, and predict therapeutic response and outcomes.

### CROSS-REFERENCE TO RELATED APPLICATIONS

This application claims the benefit of U.S. Provisional Patent Application No. 62/988,341 filed on Mar. 11, 2020, which is hereby incorporated by reference in its entirety.

### FIELD OF THE INVENTION

This invention relates to methods employed for processing in-situ (spatial) molecular data from one or more of genomics, transcriptomics, proteomics, and other related comics, including but not limited to one or more of patient metadata such as demographics, medical records, and other information, to be used to analyze biological tissues. Analysis includes statistical methods, machine learning and artificial intelligence, and the use of neural networks to create classifiers to stratify patients. Applications may include biomarker discovery and diagnostics that identify states of disease, predict response to therapies, predict disease relapse and recurrence, predict acquired drug resistance, and identify treatment strategies.

### BACKGROUND

Cancer continues to burden the global healthcare system as personalized medicine has had limited success. Targeted therapies only benefit a select population of patients amenable to those targets. This has led to high demand for biomarkers used to stratify patients into groups such as responders and non-responders. For this reason, Companion Diagnostics (CDx) are the delivery vehicle of personalized medicine, however today's CDx tests suffer from low accuracy, and patients who receive targeted therapies regularly develop acquired drug resistance to those treatments. Today's biomarkers and CDx tests are inadequate to deliver the promise of personalized medicine, and therefore new technologies and methods are required.

Initially driven by the field of Immuno-oncology, demand has been growing for technologies that integrate molecular analysis (comics) with pathology (imaging). This in-situ molecular information may provide insight into the immune response in tumors, uncover interactions within the tissue microenvironment, and resolve heterogeneity across the tissue. This has led to the emerging field of Spatial Genomics and Transcriptomics, involving the in-situ mapping of comics data across tissues. Biomarkers have historically been based on analysis of entire tissue sections, where molecular signal is averaged when cells are lumped together and analyzed as a group. However, new biomarkers are required that leverage the advances of in-situ, spatial analysis which take into account both a molecular signal, where it is located in a tissue, and its proximity to and interaction with signals around it. This new genre of biomarker, “spatial biomarkers”, may provide insights into immuno-oncology, tumorigenesis, and many more fields beyond oncology, such as developmental biology, Alzheimer's, and more. In oncology, spatial biomarkers have the potential to more accurately stratify patients into groups of responders and non-responders as well as predict recurrence, drug resistance, and more.

### SUMMARY OF THE INVENTION

In one aspect, the invention provides for the identification of biomarkers based on complex networks of signatures from tissues that may include molecular data, as well as patient metadata, such as health records and demographics. These complex biomarkers encompass details of the tissue microenvironment as well as cell-cell interactions which are crucial for more accurately describing biological systems. System-level signatures are necessary to increase the accuracy of companion diagnostic tests in immuno-oncology, as well as for other diseases and in basic research applications.

In another aspect, the invention enables analysis of spatial molecular information in a way that reduces the required computational resources compared to other methods, and enables neural networks to process spatial data while preserving macro and micro-level trends across the geography of a tissue. Classifiers built using spatial data have the potential to be used to classify disease states to identify and predict disease progression, as well as identify patient populations who may respond to treatment.

In another aspect, the invention provides strategies for augmenting spatial data to become of higher or lower resolution, as needed, in order to better characterize tissues and increase the accuracy or probability of identifying biomarkers.

Additional objects, advantages and novel features of the present invention will be set forth in part in the description which follows, and will in part become apparent to those in the practice of the invention, when considered with the attached figures.

### BRIEF DESCRIPTION OF THE DRAWINGS

Some embodiments of the invention listed in this disclosure are illustrated as pieces to exemplify the disclosure and are not limited by the figures of the accompanying drawings, in which the following references and those of the like may indicate examples of similarities to the disclosure and in which:

FIG. 1A depicts a schematic of a tissue analysis system in accordance with an exemplary embodiment;

FIG. 1 depicts a workflow whereby multiplexed spatial molecular data is generated from a tissue, an algorithm processes that data, and an output is reported, such as a disease state, prognostic indicator, response prediction, drug resistance prediction, or other classifications;

FIG. 2 depicts an exemplary hierarchical structure diagram of different potential arrays;

FIG. 3 depicts how recognition software can be applied to tissues with spatial data sets;

FIG. 4 depicts how spatial data can be down-sampled by pooling regions of data to produce lower resolution data that approximates the high-resolution data while making analysis less computationally expensive and more amenable to clinical testing;

FIG. 5 depicts how multiplexed data is structured to create a classifier to stratify patients;

FIG. 6 further depicts an embodiment of treating multiplexed spatial data as an array of images whereby each image represents a single analyte and the array collectively represents data from a tissue;

FIG. 7 depicts an output from a series of Generative Adversarial Networks (GAN) used to train classifiers and augment datasets;

FIG. 8 depicts various exemplary processes for processing spatial data including strategies to up-scale, down-scale, or compress the resolution of spatial datasets prior to analysis;

FIG. 9 depicts a study performed on a cohort of breast cancer patients where multiplexed spatial data was generated from patient tumors;

FIG. 10 depicts an embodiment of data analysis based on spatial maps of the breast cancer patients from FIG. 9 with supervised hierarchical clustering of data from specific regions of tissue from each patient produced stratified patient populations;

FIG. 11 depicts an embodiment of data analysis based on spatial maps of the breast cancer patients from FIG. 9 where gene expression trends from adjacent tissue compartments are used to stratify patients, and gene expression is visualized by graph;

FIG. 12 depicts an embodiment of data analysis based on spatial maps of the breast cancer patients from FIG. 9 where gene co-expression networks illustrate complex interdependencies between highly correlated genes from stratified patient populations, visualizing parameters that contribute to stratification between these patient cohorts;

FIG. 13 is an exemplary analysis flow in accordance with an embodiment of the present invention;

FIG. 14 is another exemplary analysis flow in accordance with an embodiment of the present invention; and

FIG. 15 is a block diagram generally illustrating a computing environment in which the invention may be implemented.

### DETAILED DESCRIPTION OF THE INVENTION

It is to be noted that the term “a” or “an” entity refers to one or more of that entity; for example, “an antibody” is understood to represent one or more antibodies. As such, the terms “a” (or “an”), “one or more,” and “at least one” can be used interchangeably herein.

Tissue analysis system 10 may generally comprise a computing device 12, which is in communication with a data storage device 14, and which optionally may be in communication with to one or more clinical devices, such as a physician's or technician's computer 16, and/or data storage device 18. The communication between the aforementioned devices may be achieved using a wireless, wired, or other type of physical connection, such as a Universal Serial Bus (USB) connector or cable, an IEEE 802.3 (Ethernet) network interface, or other suitable interface or adapter. Clinical devices may also be any type of data storage media, such as magnetic and solid state disk drives, optical media, or network file shares.

Computing device 12 is configured to run one or more software applications for performing the methods and processes described herein. As used herein, the term “software application” or “application” refers to computer-executable instructions or an algorithm stored in a non-transitory medium, such as a non-volatile memory, and executed by a computer processor. The computer processor, when executing the instructions, may receive inputs and transmit outputs to any of a variety of input or output devices to which it is coupled to perform the method described herein.

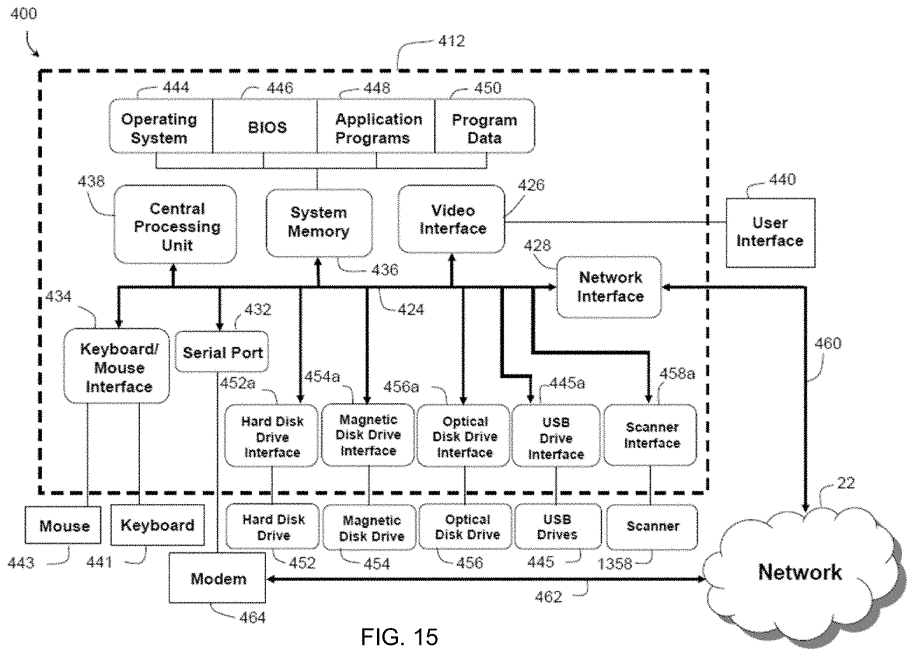

Data storage device 14 may be a non-volatile data store coupled to computing device 12. For example, data storage device 14 may be an external storage device locally coupled to computing device 12, or an internal data storage device such as a hard drive. In some cases, computing device 12 may be coupled to a networked remote data storage device or server 20 via a data communication network 22. Data communication network may be a private data communication network, such as a local area network or wide area network, or may also be a public data communication network, such as the Internet. An exemplary computing device will be described below with reference to FIG. 15.

As discussed in greater detail below, computing device 12 is provided with a software application for performing tissue analysis. In operation, the tissue analysis application can be used to retrieve the data collection, e.g., from data storage device 14, and to generate a user interface to facilitate tissue analysis of the data collection as described further herein.

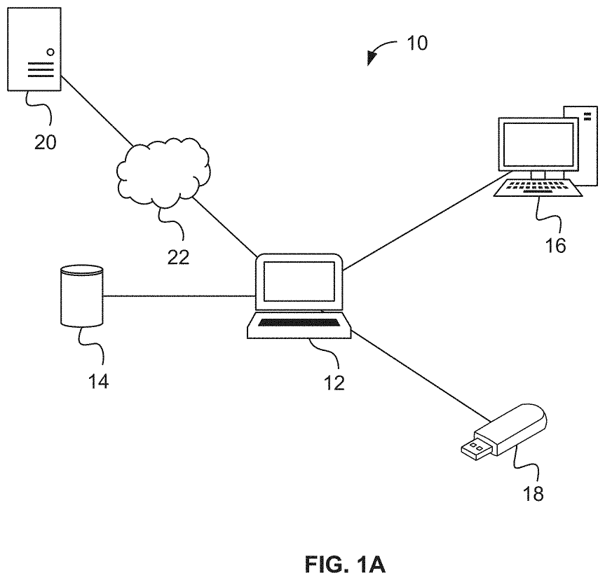

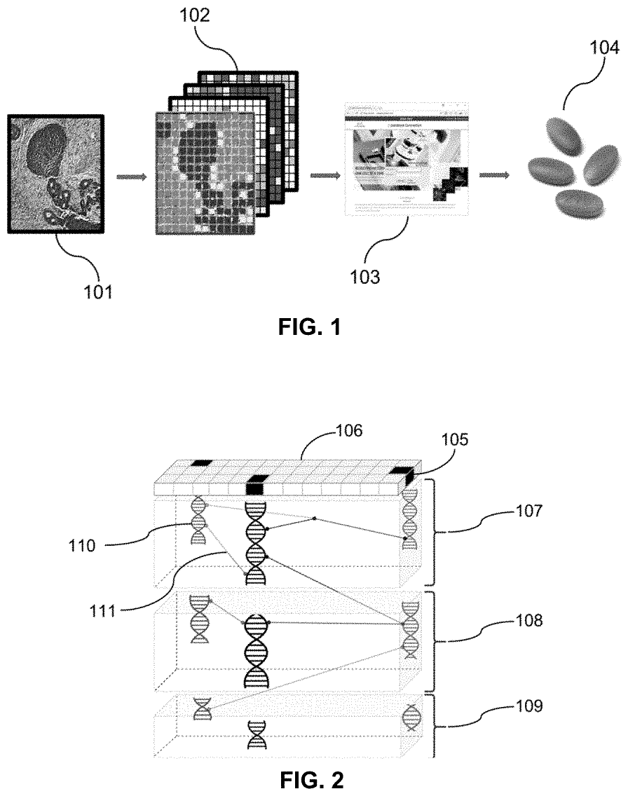

Attention is now turned to FIG. 1, which depicts a process of creating in-situ molecular information 102 from a tissue 101, analyzing that information 102, and producing a result indicating potential therapeutic response, likelihood of drug resistance, prognostic factors, and more. Tissue samples 101 may be formalin-fixed paraffin-embedded (FFPE), fresh-frozen, or fixed-frozen tissues. In-situ molecular data (spatial) 102 may be measurements of the genome, transcriptome, proteome, methylome, and other analytes. By way of example and without limitation thereto, DNA or RNA may be extracted from select cells and amplified, such as via PCR to provide transcriptomic data such as relative gene expression levels, or via a library preparation process where the library can be sequenced to provide transcriptomic or genomic data including mutations, deletions, additions, counts or FPKMs (Fragments per kilo base per million mapped reads). As discussed in more detail below, spatial data 102 may then be processed, such as by algorithm or neural network 103 which may be accessible by online portal or other method, and return a result, such as recommendations for combination treatments 104, or other relevant scores or predictions. In accordance with an aspect of the present invention, the algorithm may be a deep learning algorithm and comprise either a supervised or unsupervised neural network.

An exemplary embodiment of in-situ molecular data structured for analysis by neural networks is shown in FIG. 2. A tissue is analyzed, such as via the above examples, such that molecular measurements of analytes at arbitrary resolution span a region of the tissue or the entire tissue, thereby defining a spatial dataset 106. Individual units of spatial molecular resolution 105 define the resolution of the spatial dataset 106. Dataset 106 may contain information from many analytes 110 across multiple comics and is structured such that data on these analytes is correlated with each unit 105.

In accordance with an aspect of the present invention, each tissue sample 101 is tracked from removal from the patient to analysis. Collection software correlates the spatial location of the tissue sample 101 to a position on a collection plate or tube. This plate or tube is then tracked though the analysis process. An exemplary dataset may include data correlated to each unit 105 such as transcriptomic data 107, genomic data 108, proteomic data 109, and/or other modalities such as methylation, epigenetic, glycosylation, as well as including patient metadata such as medical records and demographic data. Markers or signatures 111 in the data 107, 108, 109 may be identified, such as via a neural network, based on correlations between locations of units 105, relative measurements, or other factors.

As will be explained in greater detail below, in one example, patient metadata is incorporated into the dataset such that, if a tissue sample has, for example, multiplexed gene data 108, the patient metadata is used to augment that gene data (for example adding the metadata to gene data 108) so that the neural network uses the metadata as another variable to train against. In an alternative example, patient metadata is incorporated with the neural network conclusions so that if a classifier identifies a genetic signature 111 within the gene data 108, the genetic signature 111 is then correlated to the patient metadata.

The neural network may then output a score based upon the identified tissue signatures. The score may be, by example and without limitation thereto, a level of heterogeneity in the tissue, entropy in the tissue or an estimate of phenotypic features based on the molecular data, including one or more of cell density, cell counts, tumor purity and cell types. It should be understood that these datasets 106, 107, 108, 109 may be hundreds of gigabytes in size with millions of variables. As such, it would be impossible for a human to inspect these datasets and identify any relational patterns. In other words, the data cannot be interpreted by the human mind.

As generally shown in FIG. 3, neural networks can be used with spatial molecular data whereby spatial data can create a unique fingerprint of a tissue for classification purposes. Nodes 201 based on molecular features can be identified in molecular data sets, such as the exemplary fluorescently labeled protein marker imaging 113, or post-analysis using the above methods, such as areas of specific mutation determined by sequencing or an area of over-expression of a certain gene as determined by PCR. It should be noted that nodes 201 may be identified in several ways. One non-limiting example may be that if a cancer-specific target is currently known (such as an infiltrating immune cell, e.g., tumor infiltrating lymphocyte (TIL)), then the known target may be designated as a first node (node 1) and additional molecular information from that location may be used to compare against the location of a second node (node 2). In this above example, node 1 may be the location of the TIL while node 2 may be the location of cancer cells.

Once nodes 201 have been identified, the nodes may be parameterized to generate unique signatures of tissues. Non-exhaustive examples of parameters may include distance between nodes, distance to other known targets, levels of genetic expression/chemical measurement, and image density/stain density from image analysis, such as but not limited to immunohistochemistry (IHC) imaging, fluorescent staining and/or hematoxylin and eosin (H&E) imaging. One non-limiting example in the field of immune-oncology may be the parameterization of the distance between a node of TIL cells (node 1, above) and a node of cancer cells (node 2, above). This parameterized distance may indicate a potential pathological response (e.g., whether TIL cells invade or stay away from cancel cells may be indicative of the body's ability to fight the cancer or response to certain drugs that stimulate the immune system).

In the case where comparison of molecular data and pathology imaging is required, molecular nodes 201 can be aligned with pathology images 114 by means of transforming the data set based on reference nodes 201 present in both the molecular data set and the imaging data set. That is, the pathology image represents the intensity of light captured by the camera across the tissue while the molecular data captured by spatial-instruments represents the intensity of the analytic target (e.g., gene expression) across the tissue. Identical reference nodes within the pathology image and molecular data are identified so that a mathematical transformation matrix can be determined. The transformation matrix may then be applied to one or the other of the pathology image or molecular data so as to align the pathology image and the molecular data with one another.

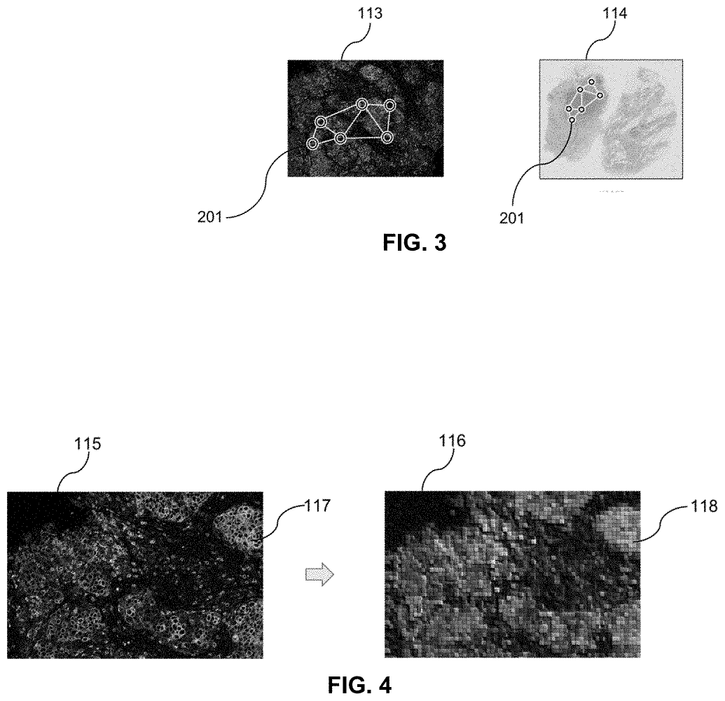

Turning now to FIG. 4, spatial data sets, exemplified here by a multiplexed immunofluorescence image 115, may be of arbitrary resolution depending on the means the data was created. In some cases, super high-resolution data may require down-sampling to make computational analysis feasible, or because clinical utility or predictive accuracy of a model requires different data resolutions. By way of example, data sets may be hundreds of gigabytes per tissue sample. It is unfeasible, even with today's most advanced computers, to load a tissue's multiple data sets into memory for processing by a neural network. Thus, lowering the resolution reduces the size of the data set thereby enabling computer processing. As shown in FIG. 4, an exemplary embodiment of down-sampling is illustrated whereby high-resolution data of a cell 117 is pooled with adjacent data such that a new composite data set 116 is created where the resolution is units of pooled spatial units 118 consisting of representative spatial data having a lower resultant resolution.

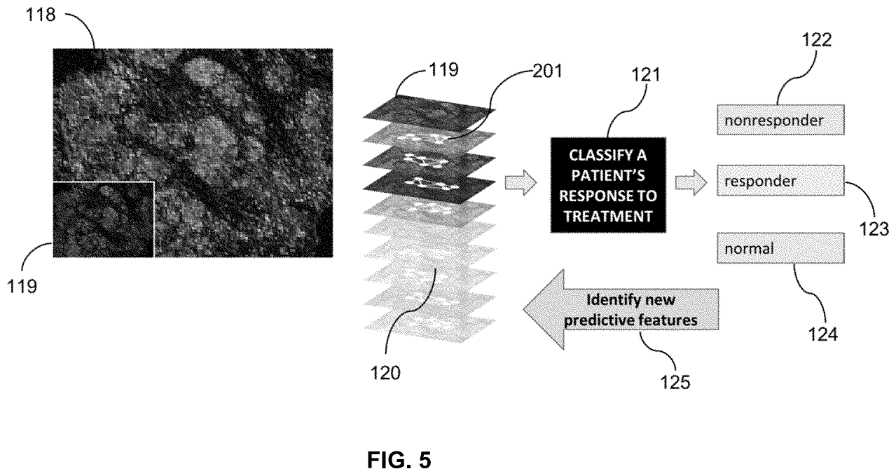

Attention is now turned to FIG. 5, whereby an exemplary embodiment of structuring spatial datasets for deep learning by neural networks. Spatial data, exemplified here by multiplexed immunofluorescence measurements 119 but may include multi-omic data previously discussed in FIG. 2, may be down-sampled by pooling 118 as previously discussed in FIG. 4. This data set is pre-processed (placed into a database/file structure so that the data is organized and optimized for input into a neural network) such that the data set is analogous to an image of various channels 119, where in-situ information for an analyte is analogous to a single channel of an image 120. A classifier may be built 121, based on neural networks or other data processing methods, that identifies biological features or nodes 201 in the analogous image channels in order to form a model which classifies patients into sub-populations such as, for example, therapeutic responders 123, non-responders 122, and healthy 124. The model may provide feedback to identify the biological features or nodes 201 that impacted classification such that new predictive features can be identified 125, as well as perform back propagation during training to automatically determine nodes 201 of predictive value.

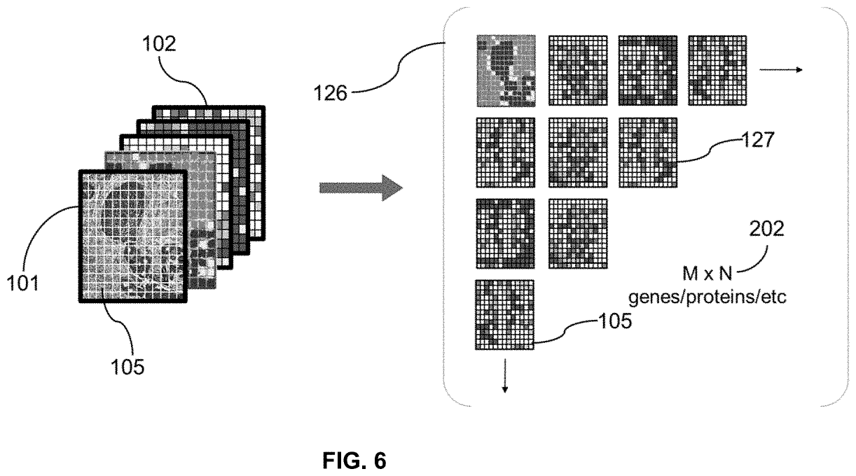

An additional exemplary embodiment of processing spatial data is shown in FIG. 6 where a tissue 101 has been analyzed, such as by one or more of the previously discussed comic methods, and in-situ molecular measurements of arbitrary units of resolution 105 have been created, whereby the data is multiplexed and treated as if there are unique maps of the same tissue for different analytes 102. Similar to the embodiment illustrated in FIG. 5 whereby each analyte represented an analogous channel of an image, here an array 126 may be created whereby a map of each analyte 102 for a tissue 101 may be organized. In the array, analyte maps 127 are arranged in a matrix of size 202 that corresponds to the number of analytes measured in the tissue. Each map preserves the location data of the resolution units of molecular analysis 105. This array 126 may be processed by neural network, or other methods, including a generative adversarial network (GAN) whereby the GAN may augment the dataset and facilitate classification by generating tissues of different classes, is discussed in greater detail below.

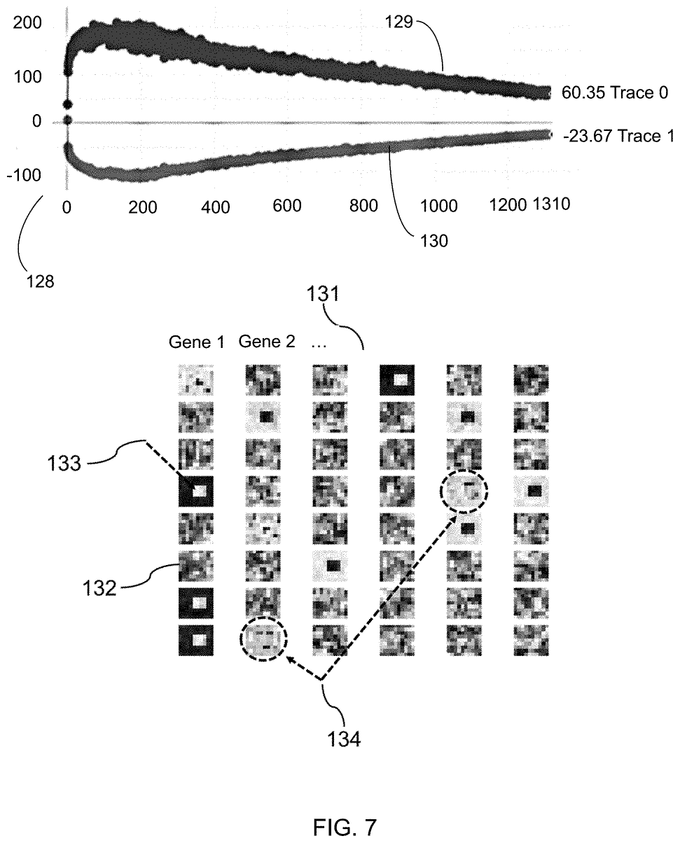

FIG. 7 expounds on the exemplary embodiment of a GAN using the pre-processing scheme of FIG. 6 whereby a sample belonging to the class of “Therapeutic Responders” 131 was generated. Here, the GAN has generated an array of multiplexed spatial data whereby each image represents a single analyte from this tissue, and regions of tumor and immune response around the tumor have been identified. Metrics of the GAN during generation are shown in graph 128 where loss between the Generator neural network 129 and Discriminator neural network 130 are graphed during training and convergence of the two indicate completion of training as the Generator 129 and Discriminator 130 are both accurately performing. In the array of generated data 131, individual exemplary maps for analytes 132 are visible and biological signatures determining this patient as a “Therapeutic Responder” are visible, as areas of tumor 133 and areas of immune response 134 around the tumor are highlighted.

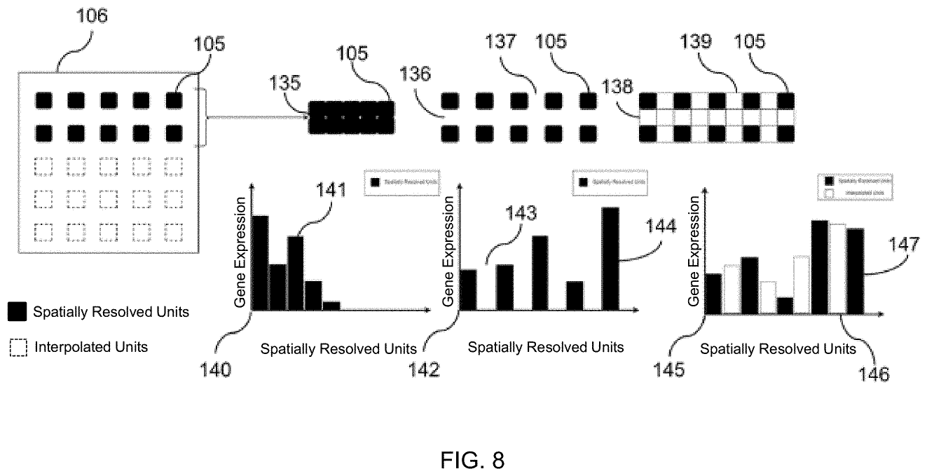

Attention is now turned to FIG. 8 which depicts various exemplary processes for processing spatial data including strategies to up-scale, down-scale, or compress the resolution of spatial datasets prior to analysis. A spatial dataset generated on a tissue sample 106 contains molecular data of units of arbitrary resolution 105. These units may be measured uniformly or randomly across a region of the tissue or spanning the whole tissue as determined by a user during the sample collection process and may be driven by the specific hypothesis in question. Depending on the method used to perform the molecular measurements, there may be discrete spaces between units 105 or there may be continuous measurements without gap across the region or tissue. For example, a tissue may be collected in its entirety and analyzed in a grid-like fashion for the entire tissue. Alternatively, specific regions of interest within the tissue may be identified by software or a pathologist for analysis (e.g., specific regions of tumor cells interspersed within healthy tissue). It should also be noted that certain collection instruments and methods require gaps between measurement sites.

In one exemplary embodiment, for measurements taken where discrete gaps between data units 105 exist, the data may be consolidated 135 such that spatially resolved units are adjacent to each other creating a virtual representation of the original region or tissue 106. Corresponding readouts of the data 140 show analytes 141 corresponding to each spatially resolved unit 105. In this case, the data is being structured during pre-processing such that the algorithm does not need to know that “gaps” exist.

Another exemplary embodiment of processing data when gaps are present between molecular measurements is to preserve those gaps in the dataset as shown in 136. Here, spatially resolved units 105 are recorded while the gaps 137 are also recorded, such that corresponding analysis and readouts of the data 142 show results of measured analytes 144 while illustrating the gaps 143. The gaps can be preserved as “zeroes” or “Not a number” to preserve distance metrics between data or nodes during analysis by the neural network, or the gaps can by synthetically eliminated and the neural network can process the data with a modified dataset.

Another exemplary embodiment of processing data when gaps are present between molecular measurements is to interpolate the data between measured units as shown in 138. Any suitable interpolation method may be used during pre-processing of the data, such as but not limited to linear, nearest neighbor or splines. Additionally or alternatively, the neural network may also perform the interpolation. By way of example, spatially resolved units 105 are recorded while gaps 137 are also recorded, and the measurement of analytes is estimated or interpolated between units 105. In this way and as shown in FIG. 8, the resolution of the spatial dataset is up-scaled, which may be reflected in the corresponding analysis. The resultant readouts of the data 145 may then show the results of measured analytes 147 as well as results of estimated values between units 146. Confidence levels for any upscaling/downscaling may also be provided to the user.

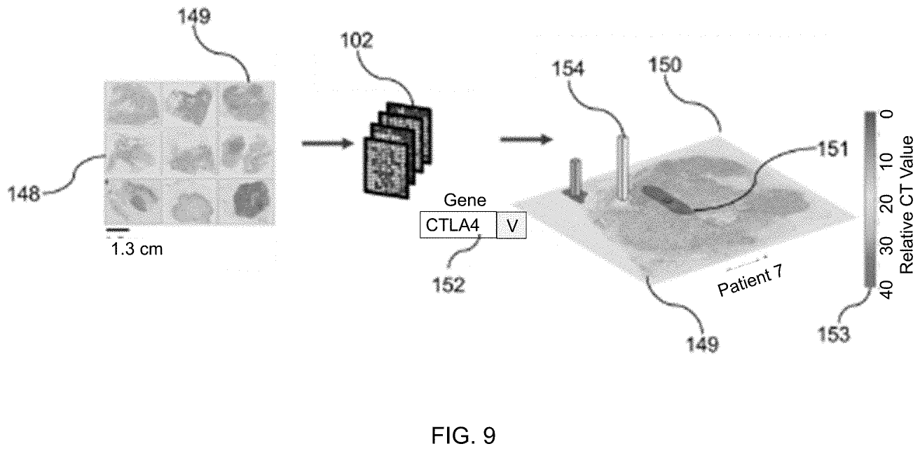

Turning attention to FIG. 9, an exemplary study was performed illustrating the application of in-situ molecular analysis and data analysis for patient stratification. Tissues from twenty-two primary breast cancer patients were analyzed 148, each with corresponding pathological imaging such as immunohistochemical (IHC) stains, H&E stains. In this example, gene expression was measured by a custom 248 gene panel by polymerase chain reaction (PCR) and expression values were measured by CT values 153. Multi-omic analysis was performed that included spatial gene expression analysis of the transcriptome, mutation analysis of the genome, Tumor Mutational Burden (TMB), and Microsatellite Instability, all of which collectively created a spatial dataset for each patient 102, as structured by methods described above and as illustrated in FIG. 2 and FIG. 5. Raw results for each patient may be visualized, such as the exemplary visualization in FIG. 9 where data from a patient 149 is visualized as a heat map 150. Regions of interest as identified by Pathologist 151 have gene expression results indicated by color and bar graph 154, and genes of interest can be chosen for visualization electronically 152.

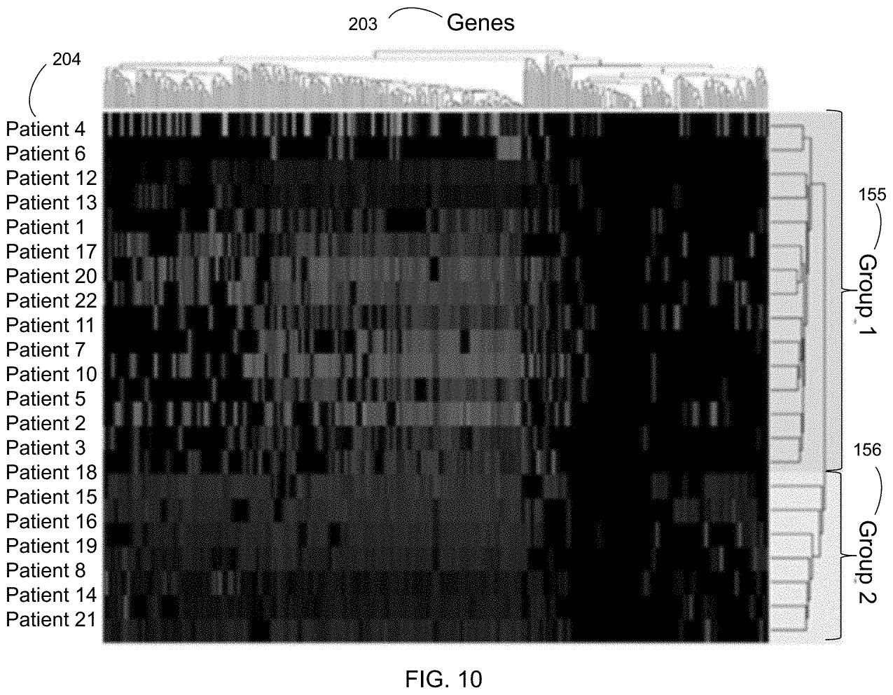

FIGS. 10, 11 and 12 generally depict embodiments of analysis performed on the spatial data generated in the study discussed in FIG. 9. FIG. 10 depicts hierarchical clustering 204 performed on data from select regions of tissues. In this instance, data from the tumor interface of all patients was pre-processed and clustered, producing stratified groups of patients with inflamed immune responses 155 and suppressed immune responses 156. Dendrograms of genes influencing clustering are also shown 203.

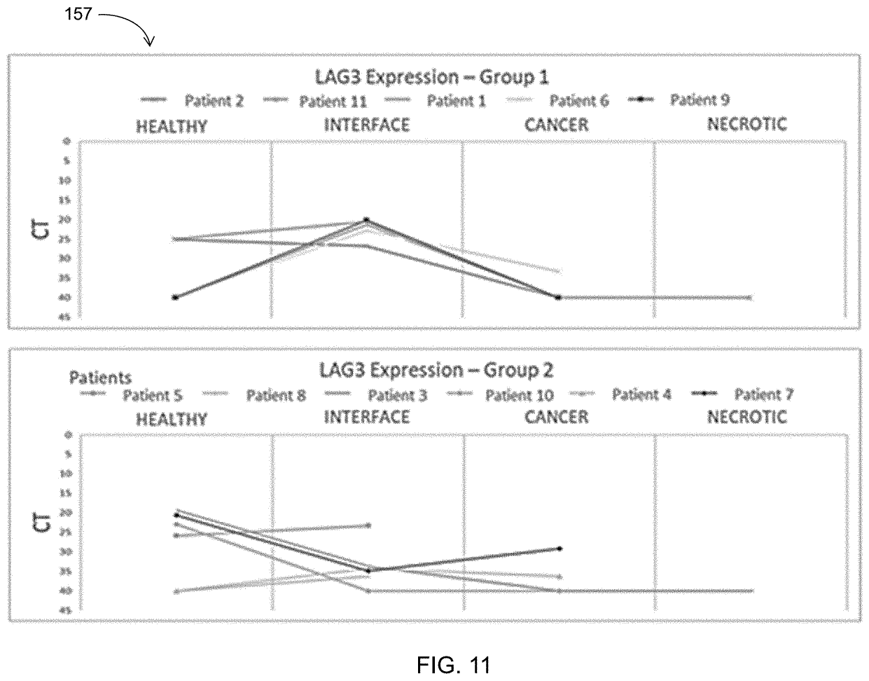

As shown in FIG. 11, this embodiment of data analysis visualizes trends across the tumor for select patients, visualizing the correlations and interactions between regions of the tissue 157. Gene expression for a key checkpoint gene, LAG3, is visually seen to change between relevant regions of the tumor, and patterns appear symmetrically opposite between stratified patient sub populations. Thus, as can be seen, the tissues have “microenvironments” and, specific to cancer, there are regions around a tumor that have unique gene signals occurring. The scientific community knows that there are signals in this microenvironment that can be used to predict what drugs a patient might respond to during treatment. This information may also potentially unlock new drug discoveries.

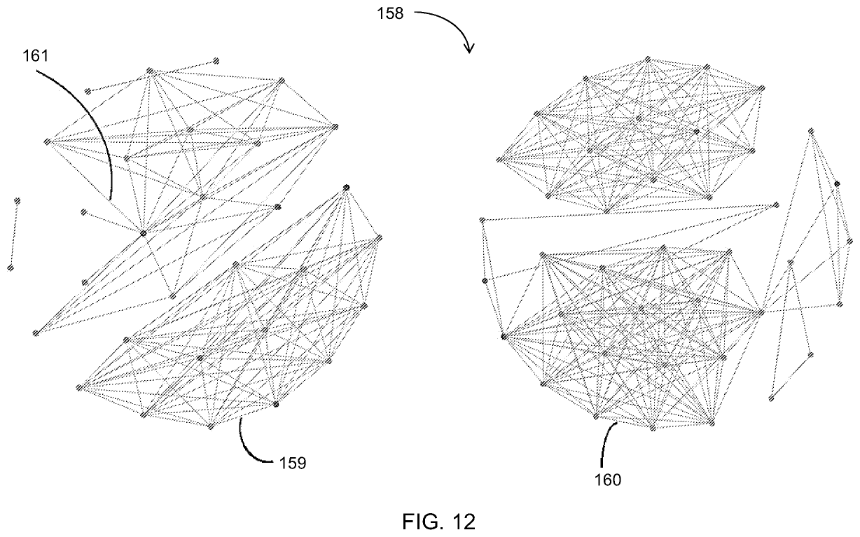

FIG. 12 depicts an embodiment of data analysis where gene co-expression networks 158 illustrate complex interdependencies between analytes from different regions of tissue. In this example, key genetic pathways were selected and screened for genes that are correlated to other genes with high R-values. Nodes represent analytes from various modalities, such as gene expression, mutation, or other metadata, and connecting lines 161 between nodes indicate statistical correlation, while line color indicates unique correlative sub-groups of nodes. These nodes are genes/analytes with their co-expression relationships identified visually. The co-expression network was determined quantitatively by screening genes against other genes and identifying genes that are related to each other (related meaning expression increases or decreases proportionally to other genes). For example, a cancer gene might increase proportionally with a DNA Repair gene, etc.

Networks of correlated genes were then plotted, specifying inverse and positive correlations along with indicating where the gene was found in the tumor microenvironment. Patients stratified by complex co-expression analysis revealed a “hot” cohort 159 and a “cold” cohort 160. “Hot” and “cold” in the context of immuno-oncology references a tumor with active or inhibited immune system response, respectively. Identifying which patients will respond positively to specific drugs is highly desired in cancer diagnostics. Thus, in accordance with an aspect of the present invention, an exemplary method described herein may be used to help distinguish between patients who may or may not respond to special drugs, thereby leading to improved patient care and patient outcomes.

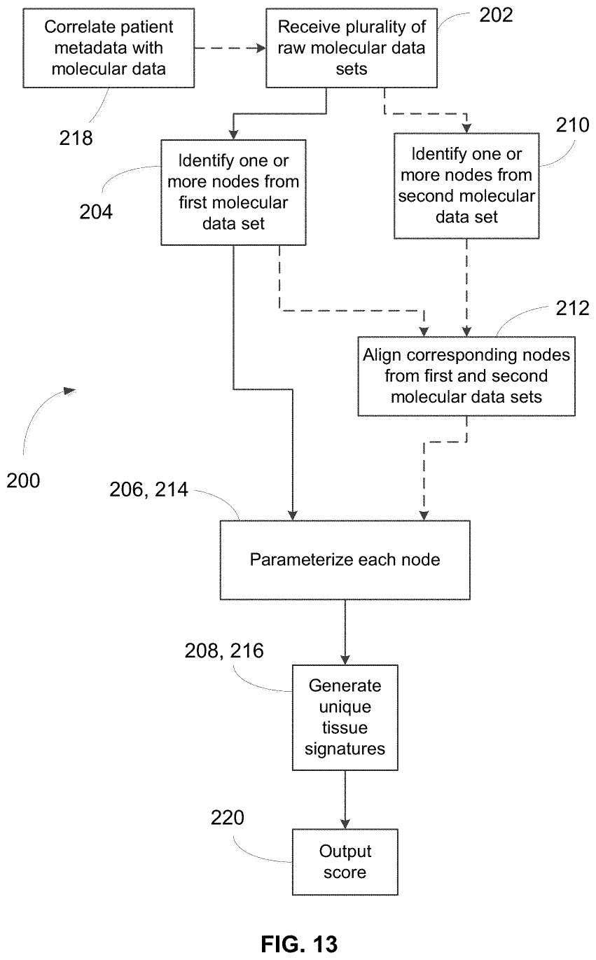

Referring now to FIG. 13, there is shown an example analytical process flow in accordance with an example embodiment of the present invention. Method 200 may be carried out, for example, using computing device 12. Method 200 starts at step 202 wherein computing device 12 receives a plurality of raw molecular data sets from the biological tissue, wherein the plurality of raw molecular data sets contain molecular data. At step 204, the tissue analysis software identifies one or more nodes within a first molecular data set of the plurality of raw molecular data sets, follow by parameterization of each of the one or more nodes within the first molecular data set at step 206. At step 208 one or more unique tissue signatures based upon the parameterized one or more nodes are generated,

As further shown in FIG. 13, method 200 may also, optionally, include step 210 wherein the tissue analysis software application identifies one or more nodes within a second molecular data set of the plurality of raw molecular data sets prior to parameterizing each of the one or more nodes within the first molecular data set. The at least one of the one or more nodes within the first molecular data set are then aligned with a corresponding at least one of the one or more nodes within the second molecular data set at step 212, followed by parameterization of each of the corresponding at least one nodes of the first and second molecular data sets at step 214. At step 216, one or more unique tissue signatures based upon the parameterized corresponding at least one nodes are generated.

In a further aspect of the present invention, method 200 may also include step 218 wherein patient metadata including one or more of medical records, medical imaging and demographic data is correlated with its respective spatial molecular data. At step 220, method 200 may also provide an output based upon the generated unique tissue signatures, where the output may be a score indicating a level of heterogeneity in the tissue, entropy in the tissue, or an estimate of phenotypic features based on the molecular data, including one or more of cell density, cell counts, tumor purity and cell types.

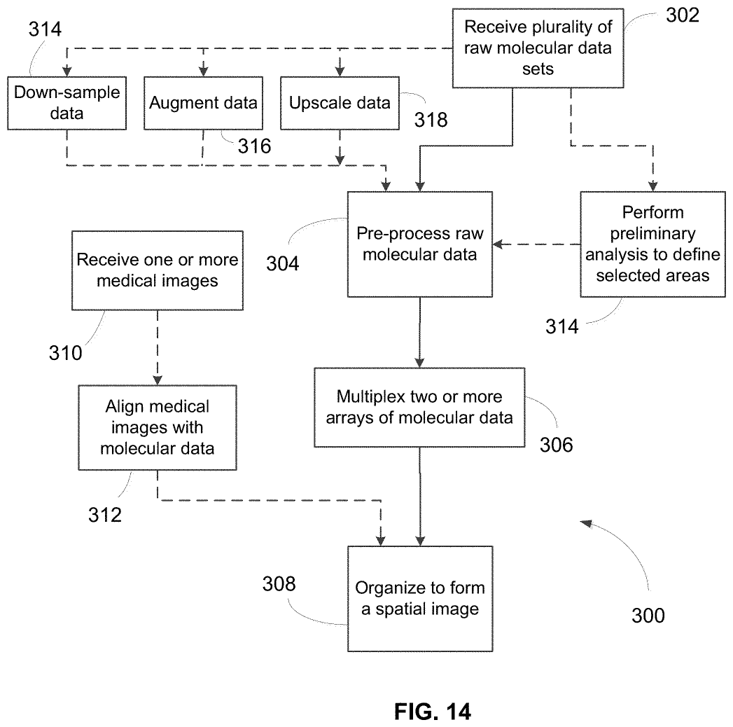

Turning now to FIG. 14, an additional exemplary analytical process flow in accordance with an example embodiment of the present invention is shown. Method 300 begins at step 302 wherein computing device 12 receives a plurality of raw molecular data sets from the biological tissue, wherein the plurality of raw molecular data sets contain spatial molecular data. At step 304, the spatial molecular data is pre-processed for analysis by a neural network, wherein the pre-processing includes creating two or more arrays of molecular data. At step 306, the two or more arrays of molecular data are multiplexed, followed by organizing the multiplexed two or more arrays of molecular data to form a spatial image at step 308, wherein spatial molecular data from one or more molecular targets represents a single channel of the spatial image.

In accordance with a further aspect of the present invention, method 300 may optionally include step 310 wherein one or more medical images of the tissue are also received by computing device 12, wherein the one or more medical images comprises tissue image data including one or more of immunohistochemistry (IHC) imaging, fluorescent staining (including fluorescent in situ hybridization, or FISH), hematoxylin and eosin (H&E) imaging, and brightfield imaging. At step 312, the molecular data is aligned with the one or more medical images.

In still another aspect of the present invention, method 300 may also include performing a preliminary analysis of the plurality of raw molecular data sets to define selected areas of spatial molecular data for pre-processing at step 314. The spatial molecular data may also be down-sampled to compress one or more of the plurality of raw molecular data sets at step 316, augmented by mathematical interpolation at step 318, or upscaled to a higher resolution by generative upscaling using the neural network at step 320.

Having described the system, processes and methods of the present invention and embodiments thereof, an exemplary computer environment for implementing the described processes and methods is provided below.

FIG. 15 shows an exemplary computing environment 400 that can be used to implement any of the processing thus far described. Computing environment 400 may include one or more computers 412 (such as computing device 12, clinical devices, physician's or technician's computer 1, server 20) comprising a system bus 424 that couples a video interface 426, network interface 428, a keyboard/mouse interface 434, and a system memory 436 (e.g., memory 14) to a Central Processing Unit (CPU) 438. A monitor or display 440 is connected to bus 424 by video interface 426 and provides the user with a graphical user interface to view certain images that may be provided by the processes and methods described herein. The graphical user interface may allow the user to enter commands and information into computer 412 using a keyboard 441 and a user interface selection device 443, such as a mouse, touch screen, or other pointing device. Keyboard 441 and user interface selection device are connected to bus 424 through keyboard/mouse interface 434. The display 440 and user interface selection device 443 are used in combination to form the graphical user interface which may allow the user to implement at least a portion of the present invention. Other peripheral devices may be connected to the remote computer through universal serial bus (USB) drives 445 to transfer information to and from computer 412. For example, cameras and camcorders may be connected to computer 412 through serial port 432 or USB drives 445 so that data may be downloaded or otherwise provided to system memory 436 or another memory storage device associated with computer 412.

The system memory 436 is also connected to bus 424 and may include read only memory (ROM), random access memory (RAM), an operating system 444, a basic input/output system (BIOS) 446, application programs 448 and program data 450. The computer 412 may further include a hard disk drive 452 for reading from and writing to a hard disk, a magnetic disk drive 454 for reading from and writing to a removable magnetic disk (e.g., floppy disk), and an optical disk drive 456 for reading from and writing to a removable optical disk (e.g., CD ROM or other optical media). The computer 412 may also include USB drives 445 and other types of drives for reading from and writing to flash memory devices (e.g., compact flash, memory stick/PRO and DUO, SD card, multimedia card, smart media xD card), and a scanner 458. A hard disk drive interface 452a, magnetic disk drive interface 454a, an optical drive interface 456a, a USB drive interface 445a, and a scanner interface 458a operate to connect bus 424 to hard disk drive 452, magnetic disk drive 454, optical disk drive 456, USB drive 445 and scanner 458, respectively. Each of these drive components and their associated computer-readable media may provide computer 412 with non-volatile storage of computer-readable instruction, program modules, data structures, application programs, an operating system, and other data for computer 412. In addition, it will be understood that computer 412 may also utilize other types of computer-readable media in addition to those types set forth herein, such as digital video disks, random access memory, read only memory, other types of flash memory cards, magnetic cassettes, and the like.

Computer 412 may operate in a networked environment using logical connections with each of the system components described above. Network interface 428 provides a communication path 460 between bus 424 and network 22, which allows data to be communicated through network 22 to and from server 22 to photofinishers computing device 412. This type of logical network connection is commonly used in conjunction with a local area network (LAN). The data related to the methods and processes described herein may also be communicated from bus 424 through a communication path 462 to network 22 using serial port 432 and a modem 464. Using a modem connection between the computer 412 and the other components of system 10 is commonly used in conjunction with a wide area network (WAN). It will be appreciated that the network connections shown herein are merely exemplary, and it is within the scope of the present invention to use other types of network connections between computer 412 and the other components of system 10 including both wired and wireless connections.

From the foregoing, it will be seen that this invention is one well adapted to attain all the ends and objects hereinabove set forth together with other advantages which are obvious and which are inherent to the device described herein. It will be understood that certain features and sub combinations are of utility and may be employed without reference to other features and sub combinations. This is contemplated by and is within the scope of the claims. Since many possible embodiments of the invention may be made without departing from the scope thereof, it is also to be understood that all matters herein set forth or shown in the accompanying drawings are to be interpreted as illustrative and not limiting.

The constructions described above and illustrated in the drawings are presented by way of example only and are not intended to limit the concepts and principles of the present invention. As used herein, the terms “having” and/or “including” and other terms of inclusion are terms indicative of inclusion rather than requirement. Further, it should be understood that the use of the terms “module” and “component” herein are interchangeable and shall have the same meaning.

While the invention has been described with reference to preferred embodiments, it will be understood by those skilled in the art that various changes may be made and equivalents may be substituted for elements thereof to adapt to particular situations without departing from the scope of the invention. Therefore, it is intended that the invention not be limited to the particular embodiments disclosed as the best mode contemplated for carrying out this invention, but that the invention will include all embodiments falling within the scope and spirit of the appended claims.

What is claimed is:

1. A method programmed for execution in a computing environment for analyzing biological tissue, utilizing a processor the method comprises:
a) receiving a plurality of raw molecular data sets from the biological tissue, wherein the plurality of raw molecular data sets contain molecular data;
b) identifying one or more nodes within a first molecular data set of the plurality of raw molecular data sets;
c) parameterizing each of the one or more nodes within the first molecular data set; and
d) generating one or more unique tissue signatures based upon the parameterized one or more nodes.

2. The method in accordance with claim 1, further comprising:
identifying one or more nodes within a second molecular data set of the plurality of raw molecular data sets prior to parameterizing each of the one or more nodes within the first molecular data set;
aligning at least one of the one or more nodes within the first molecular data set with a corresponding at least one of the one or more nodes within the second molecular data set;
parameterizing each of the corresponding at least one nodes of the first and second molecular data sets; and
generating one or more unique tissue signatures based upon the parameterized corresponding at least one nodes.

3. The method in accordance with claim 1 wherein the one or more nodes define an array of spatial molecular data.

4. The method in accordance with claim 3 wherein the spatial molecular data comprises one or more of genomic, proteomic, transcriptomic and methylomic data.

5. The method in accordance with claim 3 wherein the array of spatial molecular data is analyzed by a neural network trained to identify the one or more unique tissue signatures.

6. The method in accordance with claim 5 wherein the one or more unique tissue signatures are indicative of a disease state or are prognostic measurements of disease progression, therapeutic response, drug resistance or disease recurrence.

7. The method in accordance with claim 5 wherein one or more generative adversarial networks (GAN) is used to increase the accuracy of the neural network by training a Discriminator neural network using images created by a Generator neural network.

8. The method in accordance with claim 3 further comprising the step of correlating patient metadata including one or more of medical records, medical imaging and demographic data with the spatial molecular data.

9. The method in accordance with claim 8 wherein the medical imaging comprises tissue image data including one or more of immunohistochemistry (IHC) imaging, fluorescent staining and hematoxylin and eosin (H&E) imaging.

10. The method in accordance with claim 1 wherein steps c) and d) are conducted by a deep learning algorithm.

11. The method in accordance with claim 10 wherein the algorithm is a supervised neural network or an unsupervised neural network.

12. The method in accordance with claim 1 further comprising providing an output based upon the generated unique tissue signatures.

13. The method in accordance with claim 1 wherein the output is a score indicating a level of heterogeneity in the tissue.

14. The method in accordance with claim 1 wherein the output is a score of entropy in the tissue.

15. The method in accordance with claim 1 wherein the output is an estimate of phenotypic features based on the molecular data, including one or more of cell density, cell counts, tumor purity and cell types.

16. A method programmed for execution in a computing environment for analyzing a biological tissue, utilizing at least one processor, the method comprises:
a) receiving a plurality of raw molecular data sets from the biological tissue, wherein the plurality of raw molecular data sets contain spatial molecular data;
b) pre-processing the spatial molecular data for analysis by a neural network, wherein the pre-processing includes creating two or more arrays of molecular data;
c) multiplexing the two or more arrays of molecular data; and
d) organizing the multiplexed two or more arrays of molecular data to form a spatial image, wherein spatial molecular data for each analyte is represented as a respective single channel of the spatial image.

17. The method in accordance with claim 16 wherein the plurality of raw molecular data sets include at least one of temporal data and spatiotemporal data.

18. The method in accordance with claim 16 wherein the spatial molecular data is processed as an image using one or more of node definition, parameterization and recognition.

19. The method in accordance with claim 18 further comprising
a) receiving one or more medical images of the tissue, wherein the one or more medical images comprises tissue image data including one or more of immunohistochemistry (IHC) imaging, fluorescent staining, hematoxylin and eosin (H&E) imaging, and brightfield imaging; and
b) aligning the molecular data with the one or more medical images.

20. The method in accordance with claim 16 wherein the neural network is supervised or unsupervised.

21. The method in accordance with claim 16 further comprising defining selected areas of the spatial molecular data for pre-processing.

22. The method in accordance with claim 21 wherein one or more portions of the plurality of the raw molecular data sets are selectively loaded into a memory of a computing device based on the preliminary analysis such that spatial trends within the spatial molecular data are preserved.

23. The method in accordance with claim 22 wherein the memory is a distributed memory.

24. The method in accordance with claim 16 further comprising down-sampling of the spatial molecular data to compress one or more of the plurality of raw molecular data sets.

25. The method in accordance with claim 16 further comprising augmenting the spatial molecular data by mathematical interpolation, wherein the spatial molecular data collected from discrete locations on the tissue are used to estimate a value of spatial molecular data for a location on the tissue between the discrete locations.

26. The method in accordance with claim 16 further comprising upscaling a resolution of the spatial molecular data to a higher resolution by generative upscaling using the neural network.
